(19) United States
(12) Patent Application Publication
(10) Pub. No.: US 2016/0038452 A1
(43) Pub. Date: Feb. 11, 2016

(54) Methods for Administering a Depot of Disulfiram

(71) Applicant: Benjamin Werth, Reading, MA (US)

(72) Inventor: Benjamin Werth, Reading, MA (US)

(21) Appl. No.: 14/692,739

(22) Filed: Apr. 22, 2015

Related U.S. Application Data
(60) Provisional application No. 61/982,702, filed on Apr. 22, 2014.

(51) Int. Cl. A61K 31/27

(52) U.S. Cl. CPC A61K 31/27

(57) ABSTRACT

The present technology generally relates to a method for treating a subject, comprising administering to the subject a composition comprising a depot of disulfiram, wherein the composition is administered to the lumen of the gastrointestinal (GI) tract, the wall of the GI tract or an area that is substantially drained by the hepatic portal circulation or lacteals/lymphatic system.

### CROSS-REFERENCE TO RELATED APPLICATIONS

This application claims the benefit of U.S. Provisional Application No. 61/982,702 filed Apr. 22, 2014.

### FIELD

The present technology generally relates to a composition comprising a depot of disulfiram and methods for administering the same to a patient in order to deliver disulfiram to a desired location within a patients body, such as the gastrointestinal tact, and provide a sustained release of disulfiram over a course of weeks or months.

### BACKGROUND

Disulfiram is an orally administered drug widely used in the treatment of alcoholism. Disulfiram is one of several aldehyde dehydrogenase (ALDH) inhibitors that raise the plasma level of acetaldehyde following ethanol ingestion. The usually pleasant reaction to ethanol is thereby changed to an unpleasant one, owing to a number of bodily reactions to acetaldehyde. Anyone who consumes ethyl alcohol after pretreatment with disulfiram (taken orally) will experience the subjectively unpleasant Disulfiram-Ethanol Reaction (DER) characterized by nausea, palpitations, flushing, hyperventilation and hypotension. In theory, treatment of an alcoholic patient with disulfiram should discourage a relapse into impulsive drinking. In practice, unsupervised disulfiram therapy often ends in failure when the patient stops taking the drug and resumes drinking after the effects have worn off.

Recent reviews of disulfiram treatment have endorsed supervised disulfiram while concluding that unsupervised disulfiram administration is of limited utility [1, 2]. Since daily supervised administrations labor intensive and can cause antipathy between the supervisor and patient, an extended release version of disulfiram is desirable.

Currently used routes of administration for disulfiram depots fail to deliver suitable concentrations of disulfiram into the hepatic portal circulation. Such depot administrations are intended to discourage drinking without the need for supervised daily administration. Oral administration is generally not appropriate for depot preparations of disulfiram because a pill or solution, even if it is designed to release disulfiram over weeks to months, will pass through the digestive tract and be excreted within a relatively short time.

Subcutaneous and implanted depot preparations have been developed that are intended to release a therapeutic dose of disulfiram over an extended period of weeks to months. However, such subcutaneous implanted disulfiram tablets failed to show clinical efficacy [3-6]. Intramuscular administration has likewise proved fruitless. Patients dosed with disulfiram by deep intramuscular injection were found to have insignificant urinary levels of disulfiram metabolites compared with patients on oral disulfiram[7]. Phillips reported that patients injected subcutaneously with a fluid preparation of disulfiram developed a measurable DER when exposed to ethanol, but the subjective response was mild and only observable up to 2 weeks [6, 8, 9]. An unpublished study from Chile reports successful deterrence of drinking in patents injected subcutaneously with a formulation of disulfiram in a inclusion complex with cyclodextrin, but no disulfiram-ethanol reactions were reported [10]. No new clinical trials of depot disulfiram are known by Applicants to have been conducted in the US since the 1990's.

### SUMMARY

Described herein is a method for treating a subject, comprising administering to the subject a composition comprising a depot of disulfiram, wherein the composition is administered to the lumen of the gastrointestinal (GI) tract, the wall of the GI tract or an area that is substantially drained by the hepatic portal circulation or lacteals/lymphatic system.

### BRIEF DESCRIPTION OF THE FIGURES

FIG. 1 depicts a digram that compares the metabolism of disulfiram administered orally to that of disulfiram administered subcutaneously or intramuscularly. Disulfiram exerts its clinical effects through the inhibition of hepatic ALDH.

FIG. 2 depicts the hepatic portal vein and its territory.

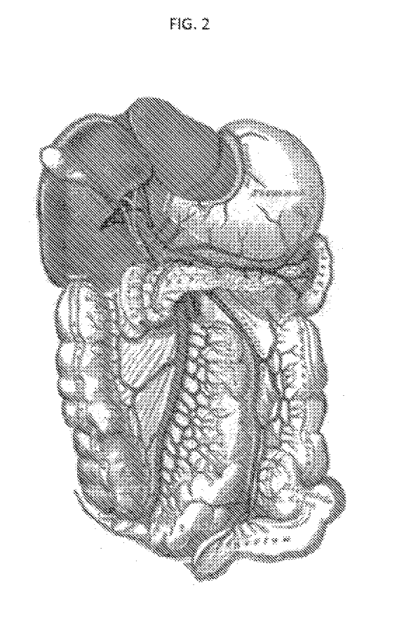

### DETAILED DESCRIPTION

In the following detailed description, reference is made to the accompanying drawings, which form a part thereof. The illustrative embodiments described in the detailed description, drawings and claims are not meant to be limiting. Other embodiments may be utilized, and other changes may be made, without departing from the spirit or scope of the subject matter presented here. The present technology is also illustrated by the examples herein, which should not be construed as limiting in any way.

### The Hepatic Portal System

In human anatomy, the hepatic portal system (also called the “portal venous system”) is the system of veins comprising the hepatic portal vein and its tributaries. The portal venous system is responsible for directing blood from parts of the gastrointestinal tract to the liver. Substances absorbed in the small intestine travel first to the liver for processing before continuing to the heart. Not all of the gastrointestinal tract is part of this system. The system extends from about the lower portion of the esophagus to the upper part of the anal canal. It also includes venous drainage from the spleen and pancreas.

Many drugs that are absorbed through the GI tract are substantially metabolized by the liver before reaching general circulation. This is known as the first pass effect. As a consequence, certain drugs can only be taken via certain routes. For example, drugs that are inactivated by the liver cannot be swallowed, but they can be taken under the tongue or transdermal (through the skin) and thus is absorbed in a way that bypasses the portal venous system. Other drugs, such as prodrugs, that need to be metabolised by the liver are best taken to be effective. The use of suppository is a way to by-pass partially the portal vein: the upper ⅓ of the rectum is drained into the portal vein while the lower ⅔ are drained into the internal iliac vein that goes directly in the inferior vena cava (thus by-passing the liver).

Blood flow to the liver includes both oxygenated and (partially) deoxygenated blood. Large veins that are considered part of the portal venous system are the hepatic portal vein, splenic vein, superior mesenteric vein and the inferior mesenteric vein. The superior mesenteric vein and the splenic vein come together to form the actual hepatic portal vein. The inferior mesenteric vein connects in the majority of people on the splenic vein, but in some people, it is known to connect on the portal vein or the superior mesenteric vein. Roughly, the portal venous system corresponds to areas supplied by the celiac trunk, the superior mesenteric artery, and the inferior mesenteric artery.

### The Gastrointestinal Lymphatic System

Lacteals are specialized lymphatic capillaries that absorbs dietary fats in the villi of the small intestine. Triglycerides are emulsified by bile and hydrolyzed by the enzyme lipase, resulting in a mixture of fatty acids and monoglycerides. These then pass from the intestinal lumen into the enterocyte, where they are m-esterified to form triacylglycerol. The triacylglycerol is then combined with phospholipids, cholesterol ester, and apolipoprotein B-48 to form chylomicrons. These chylomicrons they pass into the lacteals, forming a milky substance known as chyle. The lacteals merge to form larger lymphatic vessels that transport the chyle to the thoracic duct where it is emptied into the bloodstream at the subclavian vein. Hydrophobic drugs such as disulfiram may be incorporated into chylomicrons and transported through the circulatory system in them.

### Methods for Administering a Depot of Disulfiram

Disclosed are improved methods for administering a depot of disulfiram. Without being bound by theory, it is contemplated that the present failure of treatments with depot preparations of disulfiram might be related to how their pharmacokinetics differ from those of orally administered disulfiram. Orally administered disulfiram is absorbed by enterocytes in the GI tract and transported into the submucosa, where it is absorbed by capillaries feeding the hepatic portal vein which brings it to the liver, where it is converted to active metabolites which inhibit ALDH. Alternately, some disulfiram may find its way into the fat-transporting chylomicrons formed in enterocytes and travel through the lymphatic system to be released into the vasculature. This might increase the uptake and distribution of hydrophobic disulfiram and its metabolite diethyldithiocarbamic acid (DDC) as well as stabilizing them in the blood. Existing depot preparations are administered subcutaneously or intramuscularly, and must travel a much longer unprotected course before reaching the liver. The half-life of disulfiram in blood is only 4 minutes[13], so the site of administration is critical.

Depot preparations of disulfiram can be administered as solid, liquid, or mixed-phase formulations. A liquid or emulsification could be administered to a location by endoscopy. An endoscope with would be inserted orally or rectally and guided to the target location in the GI tract. An oral endoscope could be guided to the lower esophagus, stomach, duodenum, or jejunum. A rectal endoscope could be guided to the upper rectum or colon. An appropriate needle attachment would be used to inject the depot formulation into the GI wall. A solid depot formulation could also be administered endoscopically using appropriate surgical attachments to create a pocket in the wall of the GI tract and insert the depot.

Regarding the metabolism of disulfiram, without being bound by theory, it is contemplated that after ingestion, disulfiram is rapidly converted, probably in the stomach, to its bis(diethyldithiocarbamato) copper complex. See Scheme 1. Consequently, absorption and distribution via the gastrointestinal mucosa into the blood might involve both the parent drug and its copper complex. In the blood, both compounds are rapidly degraded to form DDC, which is unstable and is further degraded to form diethylamine and carbon disulphide. DDC is also a substrate of phase II metabolism, which involves formation of diethyldithiomethylcarbamate, (Methyl-DOC or Me-DDC) and the glucuronic acid of DDC. Me-DOC also undergoes oxidative biotransformation to diethylthiomethylcarbamate (Me-DTC), which is further oxidized to its corresponding sulphoxide and sulphone metabolites. Me-DTC may to act as a suicide inhibitor with a preference for the mitochondrial low Km isozyme of aldehyde dehydrogenases (ALDH 1), whereas the two S-oxidized metabolites, especially the sulfone metabolite, are more potent inhibitors not only of ALDH 1, but also of the cytosolic high Km isozyme of ALDH (ALDH 2).[16]

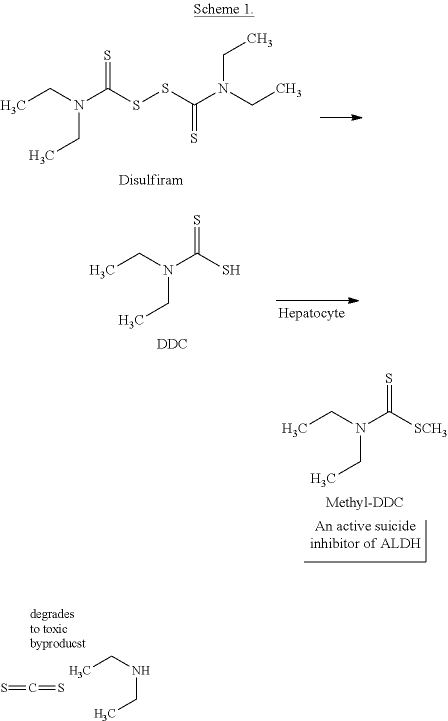

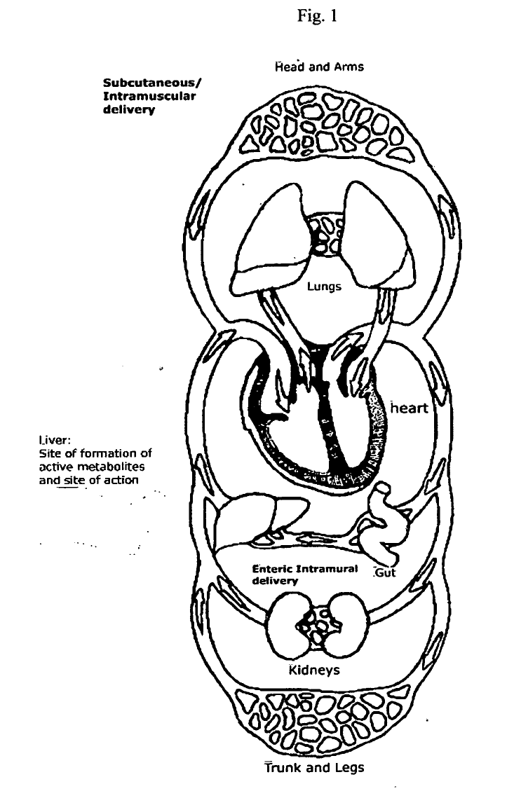

The related diagram in FIG. 1 compares the route travelled to reach the liver by disulfiram administered orally to that of disulfiram administered subcutaneously or intramuscularly. Disulfiram exerts its clinical effects through the inhibition of hepatic aldehyde dehydrogenase (ALDH).

Subcutaneous and intramuscularly administered disulfiram is metabolized to DDC, and must travel through peripheral veins, the right heart, the pulmonary arteries, pulmonary capillary bed, pulmonary veins, the left heart, aorta, and splanchnic circulation, before it reaches the liver and can be converted to its active metabolites. During the duration of this transit DDC is spontaneously degraded to the inactive metabolites carbon disulfide and diethylamine.

A depot that releases disulfiram directly into the hepatic portal circulation might result in an increased clinical effect and less toxicity. Conceivably, more DDC would reach the liver and be converted to metabolites that inhibit ALDH before being degraded to toxic metabolites such as CS₂ and diethylamine.

In some embodiments, the depot composition comprising disulfiram is injected or implanted into the mucosa, submucosa, muscularis, or serosa layer of the GI wall or between those layers of the wall of the GI tract. This could be accomplished via endoscope or a specialized swallowable device. In some embodiments, the depot composition comprising disulfiram is formulated to be taken up by the intestines. In some embodiments, the depot composition comprising disulfiram is administered via enteral or enteric administration through the gastrointestinal tract. In some embodiments, the depot composition comprising disulfiram is administered rectally. In some embodiments, the depot composition comprising disulfiram is an enterically coated oral formulation that is administered orally.

In some embodiments, the depot composition comprising disulfiram is formulated to be taken up by the lymphatic system. A depot that releases disulfiram, for example where the depot is absorbed with dietary lipids into the lymphatic system and circulation, might also result in an increased clinical effect and less toxicity. This might be because of an increased rate of absorption. This might be because disulfiram and DDC would travel through the circulation protected in a chylomicron or aggregation of short chain fatty acids, resulting in more reaching the liver before being degraded.

### Depot Pharmaceutical Compositions

Depot pharmaceutical compositions can be prepared from known protocols in the art. For example, U.S. Pat. No. 7,741,273 discloses drug depot implant designs for sustained release of therapeutic agents. These delivery designs rely on a body portion in the form of an implantable polymetric matrix for the extended release of therapeutics. Implant materials described for use in this system include, for example, hydrophilic materials, such as hydrogels, or may be formed from biocompatible elastomeric materials known in the art, including scone, polyisoprene, copolymers, of silicone and polyurethane, neoprene, nitrile, vulcanized rubber and combinations thereof. Some hydrogels are described as natural hydrogels, and those formed from polyvinyl alcohol, acrylamides such as polyacrylic acid and poly(acrylonitrile-acrylic acid), polyurethanes, polyethylene glycol, poly(2-hydroxy ethyl methacrylate) and copolymers of acrylates with N-vinyl pyrolidone, N-vinyl lactams, acrylamide, polyurethanes, and polyacrylonitrile or may be formed from other similar materials that form a hydrogel.

In some embodiments, the depot pharmaceutical compositions of disulfiram include biocompatible polymers. These polymers may include, for example, natural occurring polysaccharides, such as for example, arabinans, fructans, fucans, galactans, galacturonans, glucans, mannans, xylans (such as, for example, inulin), levan, fucoidan, carrageenan, galatocarolose, pectic acid, pectin, amylase, pullulan, chitin, agarose, keratin, chondroitan, dermatan, hyaluronic acid, alginic acid, xanthan gum, starch and various other natural homopolymer or heteropolymers such as those containing one or more of the following aldoses, ketoses, acids or amines, erythrose, threose, ribose, arabinose, xylose, lyxose, allose, glucose, mannose, gulose, idose, galactose, talose, erythrulose, ribulose, xylulose, psicose, fructose, sorbose, tagatose, manitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Exemplary semi-synthetic polymers include carboxymethylcellulose, hydroxymethylcellulose, hydroxypropylmethylcellulose, methylcellulose, and methoxycellulose. Exemplary synthetic polymers suitable for use in the present invention include polyethylenes (such as, for example, polyethylene glycol, polyoxyethylene, and polyethylene terephthalate), polypropylenes such as, for example, polypropylene glycol), polyurethanes (such as, for example, polyvinyl alcohol (PVA), polyvinylchloride and polyvinylpyrrolidone), polyamides including nylon, polystyrene, polylactic acids, fluorinated hydrocarbons, fluorinated carbons (such as, for example, polytetrafluoroethylene,) and polymethylmethacrylate, and derivatives thereof.

In other embodiments, the depot pharmaceutical compositions of disulfiram include biodegradable polymers such as, for example, poly(lactides), and poly(glycolides), poly(lactide-co-glycolides), poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polycyanoarcylates, poly(p-dioxanone), poly(alkylene oxalates), biodegradable polyurethanes, blends and copolymers thereof.

The biocompatible polymer is present in the gel vehicle composition in an amount ranging from about 5 to about 90% by weight, preferably from about 10 to about B5% by weight, preferably from about 15 to about 80% by weight, preferably from about 20 to about 75% by weight, preferably from about 30 to about 70% by weight and typically from about. 35 to about. 65% by weight of the viscous gel, the viscous gel comprising the combined amounts of the biocompatible polymer and the aromatic alcohol. The solvent will be added to polymer in amounts described below, to provide implantable or viscous gels. The aromatic alcohol enables a much wider range of polymer/solvent ratios than obtainable previously.

In some embodiments, the depot pharmaceutical compositions of disulfiram include a polymer matrix depot that breaks down over time within tissues, or which is incorporated within a protective coating to provide delayed release of the therapeutic agent. Examples of polymer matrixes include the biopolymers poly(alpha-hydroxy acids), poly(lactide-co-glycolide) (PG), polyethylene glycol (PEG) conjugates of poly(alpha-hydroxy acids), polyorthoesters, polyaspirins, polyphosphagenes collagen, starch, chitosans, gelatin, alginates, dextrans, vinylpyrrolidone, polyvinyl alcohol (PVA), PVA-g-PLGA, PEGT-PBT copolymer (polyactive), methacrylate, poly(N-isopropylacrylamide), PEO-PPO-PEO (pluronics), PEO-PPO-PAA copolymers, PLGA-PEO-PLGA, polyphosphoesters, polyanhydrides, polyester-anhydrides, polyamino acids, polyurethane-esters polyphosphazines, polycaprolactones, polytrimethylene carbonates, polydixanones polyamide-esters polyketals, polyacetals, glycosaminoglycans, hyaluronic acid hyaluronic acid ester carbonates, polydesaminotyrosine ester arylates, polyurethanes, polypropylene fumarates, polydesaminoty-rosine ester carbonates, polydesamnotyrosine ester arylates, polyethylene oxides, polyorthocarbonates, polycarbonates, or copolymers or physical blends thereof or combinations thereof. The biopolymer may also provide for non-immediate (i.e., sustained) release. Examples of suitable sustained-release biopolymers include, but are not limited to, poly(alpha-hydroxy acids), poly(lactide-co-glycolide) (PLGA), polyactide (PLA), polygycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinyl pyrrolidone, polyvinyl alcohol (PVA), PVA-g-PLGA, PEGT-PBT copolymer (polyacticve), methacrylates, poly(N-isopropylacrylamide), PEO-PPO-PEO (pluronics), PEO-PPO-PAA copolymers, PLGA-PEG-PLGA, or combinations thereof. See also U.S. Pat. No. 7,727,954.

In one aspect, provided is a depot pharmaceutical composition comprising disulfiram or a metabolite thereof or a pharmaceutically acceptable salt of a disulfiram metabolite, and at least one pharmaceutically acceptable excipient.

The depot compositions comprising disulfiram disclosed herein may be used in conjunction with any of the vehicles and excipients commonly employed in pharmaceutical preparations, e.g., talc, gum arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerin and the like.

Solid pharmaceutical excipients include starch, cellulose, hydroxypropyl cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc.

The concentration of the excipient is one that can readily be determined to be effective by those skilled in the art, and can vary depending on the particular excipient used. The total concentration of the excipients in the solution can be from about 0.001% to about 90% or from about 0.001% to about 10%.

The concentration of disulfiram can be from about 1 to about 99% by weight in the depot pharmaceutical compositions provided herein. In certain embodiments, the concentration of disulfiram in the depot pharmaceutical composition is about 5% by weight, or alternatively, about 10%, or about 20%, or about 1%, or about 2%, or about 3%, or about 4%, or about 6%, or about 7%, or about 8%, or about 9%, or about 11%, or about 12%, or about 14%, or about 16%, or about 18%, or about 22%, or about 25%, or about 26%, or about 28%, or about 30%. or about 32%, or about 34%, or about 36%, or about 38%, or about 40%, or about 42%, or about 44%, or about 46%, or about 48%, or about 50%, or about 52%, or about 54%, or about 56%, or about 58%, or about 60%, or about 64%, or about 68%, or about 72%, or about 76%, or about 80% by weight.

Depot pharmaceutical .compositions of disulfiram maybe used alone or in combination with other compounds. When administered with another agent, the co-administration can be in any manner in which the pharmacological effects of both are manifest in the patient at the same time. Thus, co-administration does not require that a single pharmaceutical composition, the same dosage form, or even the same route of administration be used for administration of both the compound of this invention and the other agent or that the two agents be administered at precisely the same time. In certain embodiments, the composition is suitable for the treatment of alcoholism.

The disulfiram can be incorporated into the depot composition in various forms, such as uncharged disulfiram, a component of molecular complexes. Acidic metabolites of disulfiram may be formulated as salts of metals, amines, or organic cations (e.g., quaternary ammonium) can be employed.

Additives which can be used in the disclosed formulations are natural and synthetic oils and fats. Oils derived from animals or from plant seeds of nuts typically include glycerides of the fatty acids, chiefly oleic, palmitic, stearic, and linolenic. Generally the more hydrogen the molecule contains, the thicker the oil becomes.

Non-limiting examples of suitable natural and synthetic oils include vegetable oil, peanut oil, medium chain triglycerides, soybean oil, almond o olive oil, sesame dl, peanut oil, fennel oil, camellia dl, corn dl, castor oil, cotton seed oil, and soybean dl, either crude or refined, and medium chain fatty acid triglycerides.

Fats are typically glyceryl esters of higher fatty acids such as stearic and palmitic. Such esters and their mixtures are solids at room temperatures and exhibit crystalline structure. Lard and tallow are examples. in general oils and fats increase the hydrophobicity of the formulation, slowing degradation and water uptake.

Another class of additives which can be used in the disclosed formulations comprise carbohydrates and carbohydrate derivatives. Non-limiting examples of these compounds include monosaccarides (simple sugars such as fructose and its isomer glucose (dextrose) disaccharides such as sucrose, maltose, cellobiose, and lactose; and polysaccarides.

Other additives, such as preservatives, stabilizers, anti-oxidants, coloring agents, isotonic agents, humectants, sequesterants, vitamins and vitamin precursors, surfactants and the like, may be added as needed. As preferred examples of preservatives, paraben derivatives are given with methyl paraben and propyl paraben given as most preferred preservatives. As preferred examples of antioxidants, butyl hydroxyanisole, butyl hydroxytoluene, propyl gallate, vitamin E acetate, and purified hydroquinone are given with vitamin E acetate and butyl hydroxytoluene given as most preferred anti-oxidants. Given as preferred examples of humectant is sorbitol. Given as preferred examples of sequesterant is citric acid.

The present technology, thus generally described, will be understood more readily by reference to the following example, which is provided by way illustration and is not intended to limit the present technology.

### EXAMPLE

### Example 1

A parenteral formulation with disulfiram, an alcohol deterrent, was made using the disulfiram (20 wt %), galactolipids from oats (48 wt %) and oil (32 wt %). The ingredients are mixed using an ultraturrax homogeniser at 2000 rpm for 15 min and at 3000 rpm for 5 min, This yields a suspension containing a high amount of finely dispersed disulfiram particles, with a smooth and homogeneous consistency. The suspension is contemplated to show excellent physical stability without significant sedimentation during storage at room temperature. The viscosity of the suspension is contemplated to be relatively low and it is possible to deliver the formulation through a syringe equipped with a thin (i,d, 1.0 mm) needle.

The formulation is applicable for instillation into the duodenum in man, where it may act as a depot for disulfiram thus providing extended alcohol deterrent effect.

### Equivalents

The present disclosure is not to be limited in terms of the particular embodiments described in this application. Many modifications and variations can be made without departing from its spirit and scope, as will be apparent to those skilled in the art. Functionally equivalent methods, processes and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, processes, reagents, compounds compositions or biological systems, which can of course vary. It is also to be understood that the terminology used herein is for the purpose of describing particular embodiments only, and is not intended to be limiting.

As used herein, “about” will be understood by persons of ordinary skill in the art and will vary to some extent depending upon the context in which it is used. If there are uses of the term which are not dear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or dearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All processes described herein can be performed in any suitable order unless otherwise indicated herein or otherwise dearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### REFERENCES

1. Buonopane, A. and I. L. Petrakis, Pharmacotherapy of alcohol use disorders. Subst Use Misuse, 2005, 40(13-14): p. 2001-20, 2043-8.
2. Anton, R. F., Pharmacologic approaches to the management of alcoholism. J Clin Psychiatry, 2001, 62 Suppl 20: p. 11-7.
3. Johnsen, J., et al., A double-blind placebo controlled study of male alcoholics given a subcutaneous disulfiram implantation. Br J Addict, 1987, 82(6): p. 607-13.
4. Johnsen, J., et al., A double-blind placebo controlled study of healthy volunteers given a subcutaneous disulfiram implantation. Pharmacol Toxicol, 1990, 66(3): p. 227-30,
5. Johnsen, J. and J. Morland, Disulfiram implant: a double-blind placebo controlled follow-up on treatment outcome. Alcohol Clin Exp Res. 1991. 15(3): p. 532-6.
6. Phillips, M. and J. Greenberg, Dose-ranging study of depot disulfiram in alcohol abusers. Alcohol Clin Exp Res, 1992. 16(5): p. 964-7.
7. Carey-Smith, K. A., et al., Assessment of intramuscular emulsified disulfiram in alcoholics by estimation of urinary diethylamine. J Stud. Alcohol, 1988. 49(6): p. 571-5.
8. Phillips, M., Persistent sensitivity to ethanol following a single dose of parenteral sustained-release disulfiram. Adv Alcohol Subst Abuse, 1987. 7(1): p. 51-61.
9. Phillips, M., Injectable formulations of disulfiram for the treatment of alcoholism, 1987, Google Patents.
10. Sepulveda, C. M. J., P. R. C. G. Von, and C. E. F. Montero, inclusion complex formed by disulfiram and a cyclodextrin, which can be used in the treatment of alcohol and cocaine dependence, 2009, Google Patents.
11. Carreño, M. J. S. and F. P. M. Gajardo, Pharmaceutical compositions containing an inclusion complex formed by disulfiram and a cyclodextrine, which can be used in the treatment of alcohol and cocaine dependence, 2011, Google Patents.
12. Carlsson, A. and B. Herslof, Lipophilic carrier preparations, 1998, Google Patents.
13. Wang, W., Disulfiram formulation and uses thereof, 2014, Google Patents.
14. Gooberman, L. L., Degradable networks for sustained release and controlled release depot drug delivery applications, 2012, Google Patents.
15. Gooberman, L. L., Implants containing disulfiram and an ant-inflammatory agent, 2010, Google Patents.
16. Johansson, B., A review of the pharmacokinetics and pharmacodynamics of disulfiram and its metabolites. Acta Psychiatr Scand Suppl,1992. 369: p. 15-26.
17. Hu, P., L. Jin, and T. A. Baillie, Studies on the metabolic activation of disulfiram in rat. Evidence for electrophilic S-oxygenated metabolites as inhibitors of aldehyde dehydrogenase and precursors of urinary N-acetylcysteine conjugates. J Pharmacol Exp Ther, 1997. 281(2): p. 611-7.

1. A method for treating a subject, comprising administering to the subject a composition comprising a depot of disulfiram, wherein the composition is administered to the lumen of the gastrointestinal (GI) tract, the wall of the GI tract or an area that is substantially drained by the hepatic portal circulation or lacteals/lymphatic system, such as the Lower esophagus, stomach, small intestine, upper rectum, or colon.

2. The method of claim 1, wherein the method yields a greater concentration of disulfiram and its active metabolites in the hepatic portal circulation relative to a concentration obtained from a subcutaneous or an intramuscular administration of disulfiram

3. The method of claim 1, wherein the method yields a concentration of disulfiram and its active metabolites in the hepatic portal circulation that is substantially similar or greater than a concentration obtained from an oral administration of disulfiram

4. The method of claim 1, wherein the disulfiram is released into the gut lumen and be exposed to pharmacologically relevant constituents of the GI tract.

5. The method of claim 1, wherein the administration comprises an injection of the composition into a vein that drains into an hepatic portal vein of the subject.

6. The method of claim 1, wherein the subject is human.
